(19) United States
(12) Patent Application Publication
(10) Pub. No.: US 2009/0018446 A1
(43) Pub. Date: Jan. 15, 2009

(54) TRANSRECTAL ULTRASOUND ABLATION PROBE

(75) Inventors: Yoav Medan, Haifa (IL); Benny Assif, Ramat Hasharon (IL); Vladimir Gershtein, Or Akiva (IL)

Correspondence Address: GOODWIN PROCTER LLP;PATENT ADMINISTRATOR, 53 STATE STREET, EXCHANGE PLACE, BOSTON, MA, 02109-2881, US

(73) Assignee: INSIGHTEC, LTD., Tirat Carmel (IL)

(21) Appl. No.: 11/775,691

(22) Filed: Jul. 10, 2007

(51) Int. Cl. A61B 8/00; A61N 7/00

(52) U.S. Cl. 600/439

(57) ABSTRACT

Transrectal ultrasound probes, systems and related methods. A probe includes an anisotropic balloon that defines a cavity for receiving a probe body that carries an ultrasound transducer. A vent tube is attached to or integral with, an outer surface of the anisotropic balloon. The balloon is structured to have inflation and elasticity characteristics that allow air bubbles between the balloon and rectal wall to be pushed away to form an improved acoustic interface while allowing bowel gas to be vented through the vent tube.

### FIELD OF THE INVENTION

The field of the invention relates generally to rectal probes and, more particularly, to transrectal ultrasound ablation probes.

### BACKGROUND

Various probe devices have been used for treatment of the prostate gland, for example, to treat prostate cancer and non-malignant prostatic hyperplasia (BPH). BPH is a well known condition that involves expansion of prostate tissue that may cause obstruction of the urethra. This may result in increased frequency of urination and slow or interrupted urinary streams and other complications.

FIG. 1 generally illustrates the anatomical structure of a male pelvic region and the location of the prostate 16 in relation to the urethra 10 and rectum 22. The urethra 10 is a duct leading from a bladder 12, out through an opening 14, through the prostate 16, and out an orifice 18 of the end of a penis 20. Growth of the prostate 16 around the urethra 10 results in application of pressure or constriction around the urethra 10, which can interrupt flow of urine from the bladder 12 and through the prostate 16, which is adjacent to the rectum 22.

Referring to FIG. 2, a known system and method for treatment of the prostate 16 involves two probes—a transurethral ablation probe 30 and a transrectal temperature probe 40. A known transurethral ablation probe 30 includes a catheter 32 that carries an ablation element 34, such as a microwave antenna. The catheter 32 and microwave antenna 34 are inserted through the urethra 10 and positioned so that the microwave antenna 34 is adjacent the prostate 16. A known transrectal temperature probe 40 in the form of a balloon 42 includes a temperature measurement or sensing device 44. The temperature measurement device 44 is inserted into the rectum 22 and positioned so that the temperature sensing device 44 is in contact with the inner surface 24 of the rectum 22.

Following positioning of the probes 30 and 40, the microwave antenna 34 is energized and radiates microwave energy to heat and necrose adjacent prostate 16 tissue. For this purpose, the temperature of prostate 16 tissue is raised to a sufficiently high temperature, e.g., above about 45° C., for a sufficient time. The temperature measurement device 44 of the transrectal temperature probe 40 is held against the inner wall 24 of the rectum 22, e.g., when the balloon 42 is inflated, to monitor the temperature of the rectum wall 24 during ablation of tissue by the transurethral ablation probe 30. A cooling fluid can be delivered through the transrectal temperature probe 40 in order to cool the rectum 22 and avoid collateral tissue damage caused by ablation of prostate 16 tissue. In one known device, the temperature measurement device 44 is integrated within the expandable balloon 42, i.e., within the body of the balloon 42 or the balloon 42 material itself. Thus, the temperature measurement device 44 can move with the balloon 42 as the balloon 42 expands and contracts. In these types of devices, the interior volume of the balloon 42 may be filled with cooling fluid. Examples of known transurethral ablation probes 30 and transrectal temperature probes 40 are described in U.S. Pat. Nos. 5,335,669; 6,009,351 and 6,348,039, the contents of which are incorporated herein by reference.

While known transurethral microwave ablation probes 30 and transrectal temperature probes 40 have been used effectively in the past, these devices and treatment techniques necessarily involve use of two separate probes. Multiple probes can complicate procedures and can increase patient discomfort. Additionally, use of microwave ablation elements may not be desirable since microwave energy may radiate from the antenna 34 in various or all directions, thus subjecting surrounding healthy tissue to microwave energy, resulting in collateral tissue damage.

Referring to FIG. 3, it is also known to use a high intensity focused ultrasound (HIFU) probe 50 that includes an ultrasound transducer 52 that emits ultrasound or acoustic energy 54 for treatment of BPH and other prostate 16 conditions. One example of a known HIFU system is described in U.S. Pat. No. 5,676,692, the contents of which are incorporated herein by reference.

During use, the distal end of the ultrasound probe 50 is inserted into the rectum 22, and the transducer 52 is activated to emit ultrasound energy 54. It should be understood that a probe, such as probe 50, is usually larger in diameter inside the rectum than the diameter at the anus. Thus, figures are provided for purposes of general illustration. The ultrasound energy 54 is focused at target prostate 16 tissue to heat and necrose selected portions of tissue. In this manner, HIFU ultrasound probes 50 provide targeted therapy by focusing energy 54.

Referring to FIG. 4, one known HIFU ablation probe 50 includes a probe body 56 that carries the transducer 52. The probe body 56 is placed inside of a balloon 58, and the assembly of the probe body 56 and balloon 58 is inserted into the rectum 22. The balloon 58 is inflated or expanded outwardly (indicated by arrows) towards the inner wall 24 of the rectum 22. An ultrasound gel (not shown) may be applied around the balloon 58 to provide desired acoustic coupling between the probe and the inner wall 24. The balloon 58 is filled with water that is circulated, chilled and degassed to protect the inner wall 24 from heat damage and for acoustic coupling.

The transducer 52 is activated and the resulting focused acoustic energy 54 heats and necroses prostate tissue. Thus, with a HIFU probe, ablation energy 54 can be delivered from the rectum 22 to target prostate tissue rather than from the urethra 10 to target prostate tissue. Further, acoustic energy can be focused and emitted in a particular direction and location rather than emitted in multiple directions as the case with microwave antennas 34.

While known transrectal HIFU probes 50 have been used effectively in the past and provide certain advantages over microwave-based systems, they can be improved. For example, referring to FIG. 5, use of known transrectal HIFU probes 50 may result in trapping of air bubbles 60 between the outer surface 59 of the balloon 58 and the inner wall 24 of the rectum 22. This may be caused by the isotropic elasticity or expansion of the balloon 58 (as shown in FIG. 5) used in known probes 50.

Trapped air bubbles 60 can cause a number of problems during treatment. For example, bubbles 60 may interfere with or block acoustic energy 54 emitted by the transducer 52. Consequently, all of the required acoustic energy 54 may not be applied to the prostate 16, thereby reducing the effectiveness of the procedure, possibly resulting in longer or multiple procedures, or having to reposition or reconfigure the probe 50 in an attempt to eliminate or displace trapped bubbles 60. Trapped air bubbles 60 may also cause acoustic energy 54 to reflect back on the transducer 52. This may result in acoustic energy 54 that adversely affects the rectum 22. Ultrasound gel that is often applied around the balloon 58 may present additional complications since air bubbles 60 are readily trapped within the gel. Additionally, air bubbles can be difficult to remove due to limited access and difficulties in identifying the air bubbles 60. Thus, bubbles 60 in the interface between the balloon 56 and the inner wall 24 of the rectum 22 can limit or inhibit effective use of HIFU ultrasound probes 50.

During a procedure, it is also desirable to prevent movement of the prostate 16, e.g., caused by discharge of bowel gas 62 through the rectum 22. However, when the balloon 58 is inflated, the balloon may seal the rectum 22, thereby blocking bowel gas 62 from passing through rectum 22, as generally illustrated in FIG. 5. The resulting accumulation of gas 62 inside the rectum 22 can cause the prostate 16 to move. Pressure from accumulated gas 62 may also cause patient discomfort, causing the patient to move or shift in order to alleviate the discomfort, thereby resulting in movement of the prostate 16.

Thus, it is desirable to have a transrectal ultrasound ablation probe that can eliminate or reduce air bubbles that would otherwise be trapped between an outer surface of a known balloon and an inner rectal wall in order to provide a more consistent interface, improve acoustic coupling and reduce other adverse effects. It is also desirable to vent bowel gas from within the rectum to the body exterior in order to reduce bowel gas buildup and reduce prostate movement and patient discomfort.

### SUMMARY

According to one embodiment, a transrectal ultrasound probe includes a probe body carrying an ultrasound transducer, an anisotropic balloon member, and a tube. The anisotropic balloon member defines a cavity configured for receiving the probe body, and the tube is associated with an outer surface of the anisotropic balloon member. The tube is configured for venting of rectal gas.

According to another embodiment, a transrectal ultrasound probe includes a probe carrying an ultrasound transducer for treating or ablating tissue, a balloon member, a tube, and a temperature sensor. The balloon member defines a cavity configured for receiving the probe body. A thickness of a middle portion of the balloon member is less than a thickness of balloon member portions adjacent the middle portion. The tube is conformable with an outer surface of the balloon member and may be used to vent rectal gas. The temperature sensor is associated with the ultrasound transducer.

Another embodiment is directed to a method of ablating tissue of a patient that includes inserting a probe into a rectum of the patient. The probe has a probe body carrying an ultrasound transducer, a balloon member defining a cavity configured for receiving the probe body, and a tube conformable with an outer surface of the balloon member. The method further includes activating the ultrasound transducer and ablating tissue using ultrasound energy emitted by the ultrasound transducer. The method may further include venting rectal gas through the tube and outside of the patient. The method may also include venting air from within the balloon member through a duct defined by a distal end of the balloon member.

In one or more embodiments, the transducer is a focused ultrasound transducer that includes an array of ultrasound transducer elements. The probe body may define a window for the ultrasound transducer, and cover element may be rotatably positionable within the probe body to expose and cover the ultrasound transducer.

In one or more embodiments, different portions of the anisotropic balloon member having different thicknesses. For example, a thickness of middle portion of the anisotropic balloon member may be less than a thickness of portions of the anisotropic balloon member adjacent the middle portion, and the middle portion may expand more than the adjacent portions when the anisotropic balloon member is inflated.

In one or more embodiments, the tube is attached to the outer surface of the anisotropic balloon member and is conformable to a shape (e.g., non-linear shape) of the anisotropic balloon member. Embodiments may be configured so that no portion of the tube is positioned inside the anisotropic balloon member. A distal end of the anisotropic balloon member may also define a duct for venting air from within the anisotropic balloon member.

In one or more embodiments, a probe may include a temperature sensor associated with the ultrasound transducer, but which is not attached to the anisotropic balloon member. Further, a probe may include location sensor that may include magnetic resonance micro-coils.

Other aspects and features of the embodiments will be evident from reading the following description of the embodiments.

### BRIEF DESCRIPTION OF THE DRAWINGS

Referring now to the drawings in which like reference numbers represent corresponding parts throughout and in which:

FIG. 1 is a sectional view of a male patient's pelvic region;

FIG. 2 generally illustrates a known multi-probe system for treating the prostate including a transurethral ablation probe and a transrectal temperature probe;

FIG. 3 generally illustrates a known system for treating the prostate and that includes a transrectal ultrasound ablation probe;

FIG. 4 further illustrates a known transrectal ultrasound ablation probe within an isotropic balloon;

FIG. 5 illustrates isotropic expansion of the balloon shown in FIG. 4 and resulting trapping of air bubbles between the outer surface of the isotropic balloon and the inner surface of the rectum to seal;

FIG. 6 illustrates a focused ultrasound ablation probe and system according to one embodiment that includes a probe body positioned within an inflated anisotropic balloon;

FIG. 7 further illustrates a probe body for use with embodiments;

FIG. 8 illustrates a probe body within a deflated or partially inflated anisotropic balloon inserted within a rectum according to one embodiment;

FIG. 9 shows how inflation of the anisotropic balloon displaces air bubbles from the center of the anisotropic balloon and sideways away from the center of the balloon according to one embodiment;

FIG. 10 shows how the ultrasound ablation probe shown in FIG. 9 can also be used for venting of bowel gas according to one embodiment;

FIG. 11 further illustrates how the ultrasound ablation probe shown in FIGS. 8 and 9 can be used for venting of bowel gas and venting of air from air bubbles displaced by the anisotropic balloon according to another embodiment;

FIG. 12 is a cross-sectional view of a deflated or partially inflated balloon illustrating different balloon portions configured for anisotropic expansion when inflated;

FIG. 13 illustrates the anisotropic balloon illustrated in FIG. 12 when inflated;

FIG. 14 illustrates another embodiment of an anisotropic balloon including hygienic shield and a vent line associated with an outer surface of the balloon and extending along a length of the balloon;

FIG. 15 illustrates another embodiment of an anisotropic balloon including an air release port at a distal tip of the balloon for bleeding or releasing air from the interior of the balloon;

FIG. 16 illustrates another embodiment of an anisotropic balloon including an external gas venting tube and an air release port at a distal tip of the balloon;

FIG. 17 illustrates a probe shell or housing of a probe body that carries an ultrasound transducer for use with embodiments;

FIG. 18 illustrates a circuit board or substrate for carrying an ultrasound transducer for use with embodiments;

FIG. 19 illustrates a shaft for carrying the probe shell and circuit board shown in FIGS. 17 and 18; and

FIG. 20 illustrates a probe body including a rotatable shield for covering an ultrasound transducer positioned inside a probe shell or housing.

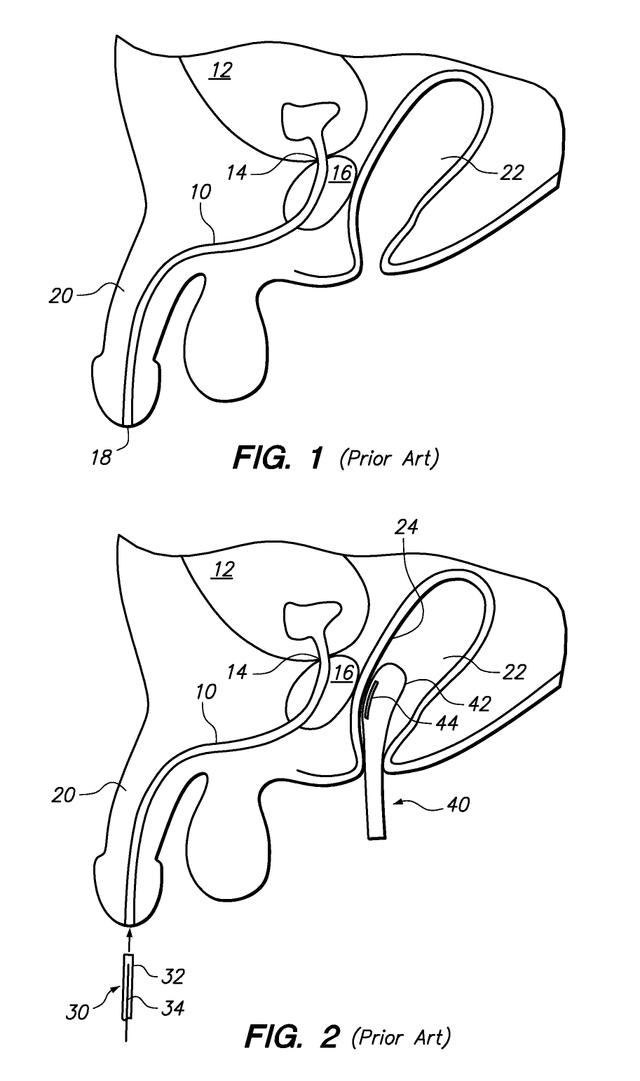

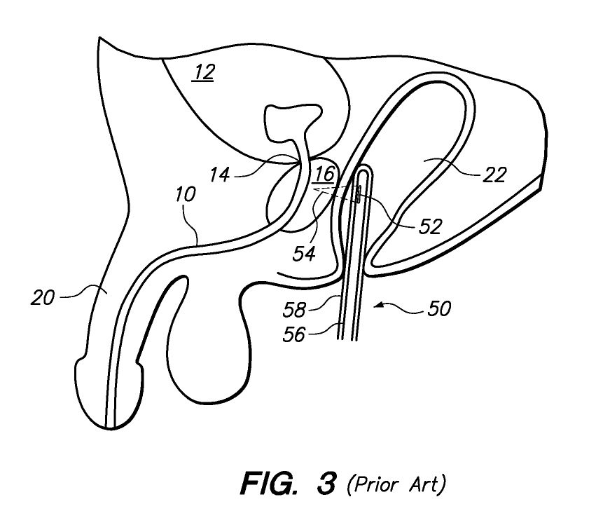

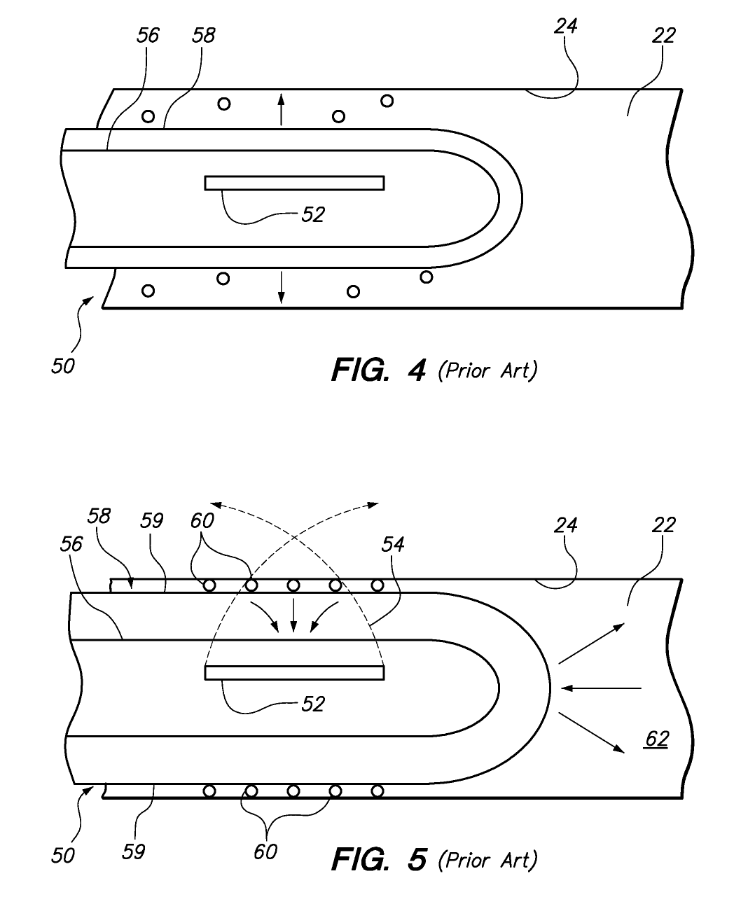

### DETAILED DESCRIPTION OF ILLUSTRATED EMBODIMENTS

Embodiments are directed to transrectal ultrasound or HIFU ablation probes, ablation systems and methods for improving acoustic coupling by eliminating or reducing air bubbles between an outer surface of a balloon and an inner wall of a rectum while venting bowel gas from within the rectum during treatment. Embodiments achieve these advantages by use of an anisotropic balloon or condom that is placed over a distal end of an ultrasound probe body or housing, and an external vent tube or duct associated with an outer surface of the anisotropic balloon.

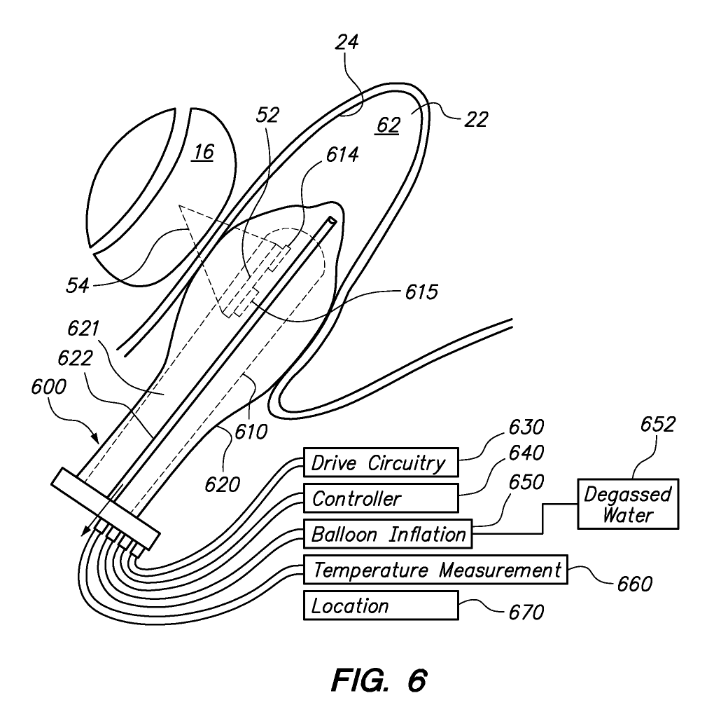

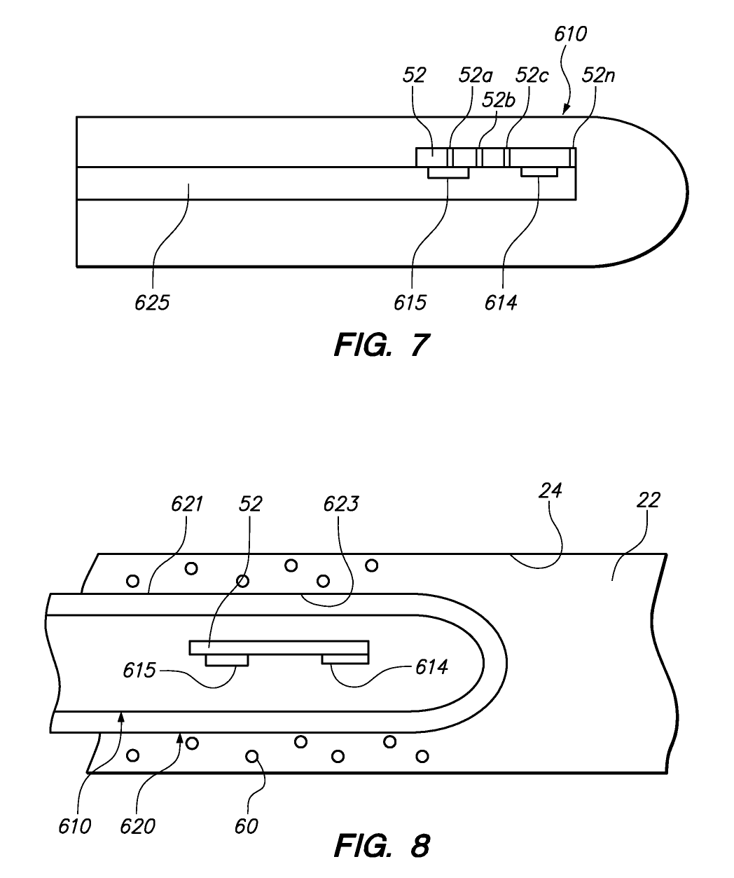

Referring to FIGS. 6 and 7, a transrectal ultrasound probe 600 according to one embodiment includes a probe body 610 (illustrated in phantom in FIG. 6) and an anisotropic balloon or condom member 620 (generally referred to as anisotropic balloon 620) that is applied over a distal end of the probe body 610. The probe body 610 carries an ultrasound transducer 52 (illustrated in phantom in FIG. 6) and associated electrical components, such as a circuit board 625 to which the transducer 52 is mounted for controlling and driving the transducer 52. The transducer 52 may be an array of multiple ultrasound transducer elements (52a-n), which may be individually controllable in order to achieve proper focusing of acoustic energy 54.

A temperature measurement device or sensor 614 and one or more location sensors 615 are associated with, e.g., coupled to, the transducer 52 or the circuit board 625. In the illustrated embodiment, the temperature sensor 614 and the location sensor 615 are coupled to an underside of the circuit board 625, but other configurations may be utilized as necessary. Further, in the illustrated embodiment, the temperature and location sensors 614, 615 are inside of the probe body 610, and are not associated with, attached to, or part of the anisotropic balloon 620 or balloon 620 material.

The temperature sensor 614 is used to monitor the temperature of the transducer 52 to ensure that the temperature of the transducer 52 does not exceed a threshold temperature. The temperature sensor 614 may also be used to monitor ambient temperature to verify the cooling effect of water inside the probe body 610 and anisotropic balloon 620 and that circulates around the transducer 52.

The location sensor 615 may also be used in order to accurately track the position of the transducer 52. According to one embodiment, the location sensor 615 may include one or more micro magnetic resonance imaging (MRI) coils that are used to localize the transducer 52 in the MRI image space. According to one embodiment, multiple micro-coils, e.g., four micro-coils, may be mounted on the underside of the circuit board 625. The probe 600 may include non-magnetic materials that are compatible with magnetic resonance imaging in order to reduce or minimize imaging artifacts.

The transrectal ultrasound probe 600 can be driven and controlled using ultrasound ablation system components including suitable drive circuitry 630, which provides electrical drive signals to the transducer 52 through the circuit board 625 or other suitable substrates or connectors. A controller 640 controls drive signals that are provided by the drive circuitry 630. The anisotropic balloon 620 can be inflated by balloon inflation element 650 that utilizes a source of degassed water 652 or other suitable fluid or material suitable for acoustic coupling. A temperature measurement element 660 can determine or output the temperature sensor 614 within the probe body 610, and a location device or system 670 can be used to monitor the position of the transducer 52 using the location sensor 615. Circuit board 625, drive circuitry 630, controller 640, balloon inflation 650, temperature measurement 660 and location or MR 670 devices are well known and, therefore, are not described in further detail.

In embodiments, the balloon 620 is anisotropic in that the elasticity and/or expansion of the balloon 620 varies along the length of the balloon 620. More particularly, in embodiments, an “anisotropic” balloon 620 has certain sections or portions that expand or inflate whereas other sections or portions do not, certain sections or portions that expand at different rates, and/or certain sections or portions that expand to different degrees or diameters. For example, in the embodiment illustrated in FIG. 6, when the anisotropic balloon 620 is inflated, a middle portion of the balloon 620 expands before adjacent end portions of the balloon 620, and/or to a larger diameter or dimension compared to adjacent end portions of the balloon 620, which may or may not be expandable.

A vent duct or vent tube 622 (generally vent tube 622) is associated with an outer surface 621 of the anisotropic balloon 620 to allow venting of bowel gas 62 in the rectum 22. Venting of bowel gas 62 prevents accumulation of gas 62 inside the rectum 22 in order to enhance patient comfort and reduce pressure on the prostate 16, thereby reducing or eliminating movement of the prostate 16 due to bowel gas 62. Although this specification refers to a “tube,” it should be understood that a “tube” can have various shapes, including circular shapes. Further, a “tube” can be a conduit, aperture, or other cavity through which a gas or fluid, such as bowel gas 62, can pass when the ultrasound ablation probe 600 is positioned inside the rectum 22 of a patient.

In one embodiment, the vent tube 622 is attached (e.g., adhered) to the outer surface 621 of the anisotropic balloon 620. In another embodiment, the vent tube 622 is integral with the outer surface 621 of the balloon 620. In one embodiment, the vent tube 622 is embedded within or formed with the material forming the anisotropic balloon 620 and extends along the length of the balloon 620 to allow venting of gas 62. For example, the vent tube 622 can be molded within or to be a part of the balloon 620 material. Thus, the proximal and distal ends of the vent tube 622 are exposed to allow venting of gas, whereas middle portions of the vent tube 622 may or may not be visible depending on the manner in which the vent tube 622 is associated with the outer surface 621 of the balloon 620.

The vent tube 622 is preferably flexible and conformable to the shape of the outer surface 621 of the anisotropic balloon 620 so that the vent tube 622 can bend with or assume the shape of a curved outer surface 621 of the balloon 620. Further, the vent tube 622 can conform or bend as the anisotropic balloon 620 is inflated and deflated. The flexibility of the balloon 620 and vent tube 622 also facilitates insertion of the probe into the rectum 22. Although FIG. 6 illustrates a vent tube 622 extending along a length of the anisotropic balloon 612, the vent tube 622 may be shorter or longer while still being capable of venting gas 62. For example, the vent tube 622 may extend from the distal end of the balloon 620 and be sufficiently long so that it extends outside of the patient and beyond the anus.

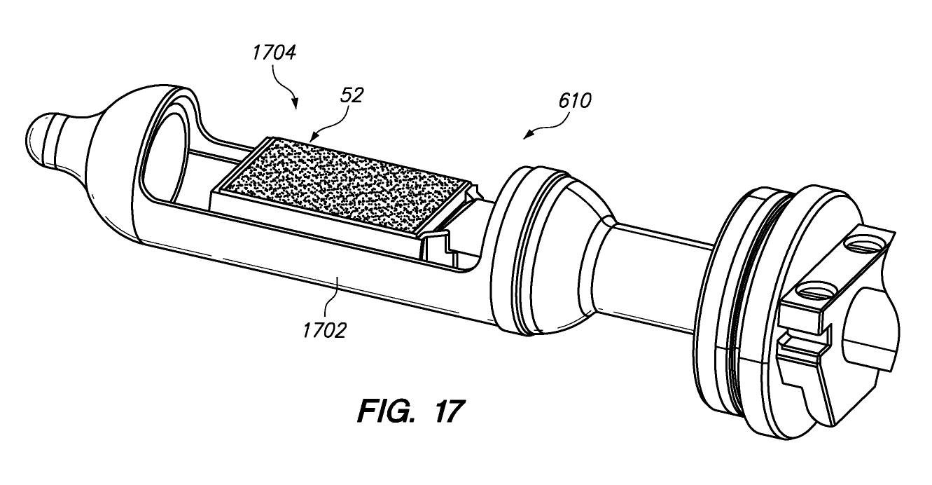

Following is a more detailed description of components of probe embodiments and how embodiments may be used for treatment of the prostrate 16 with reference to FIGS. 8-20. FIGS. 8-13 further illustrate how embodiments that use an anisotropic balloon 620 provide advantages over known ultrasound probes that use isotropic balloons, and one manner in which the anisotropic balloon 620 may be made. FIGS. 14-16 further illustrate embodiments of a balloon 620 having vent tubes 612 associated with an outer surface 621 of the anisotropic balloon 620 may be used to vent gas 62 from the rectum 22 and having a release vent or valve for bleeding air from within the anisotropic balloon 620. FIGS. 17-20 are renderings a probe body 610 and components thereof for use in various embodiments.

Prior to insertion, the anisotropic balloon 620 is placed over a distal end of the probe body 610. The anisotropic balloon 620 may initially be filled with a small amount of water to displace the area inside the anisotropic balloon 620 between the transducer 52 and the inner surface 623 of the balloon 620 to verify that the balloon 620 is free of leaks.

Referring to FIG. 8, the anisotropic balloon 620/probe body 610 assembly may be inserted into the patient through the rectum 22. An ultrasound gel may be applied around the outer surface 621 of the anisotropic balloon 620 to facilitate insertion of the probe 600 into the patient and to provide desired acoustic coupling. FIG. 8 illustrates the anisotropic balloon 620 in a partially or completely deflated state (e.g., when using a small amount of water to test for leaks) and positioned inside the rectum 22. Air bubbles 60 may exist between the outer surface 621 of the deflated balloon 620 and the inner surface 24 of the rectum 22 (e.g., within the ultrasound gel).

It should be understood that FIG. 8 is provided to generally illustrate the presence of air bubbles 60 in the space between the anisotropic balloon 620 an the inner surface 24 of the rectum 22, and that the dimensions illustrated in FIG. 8 may not accurately reflect actual dimensions. For example, the transrectal ultrasound probe 600 may be configured so that the outer surface 621 of the anisotropic balloon 620 contacts the inner surface 24 of rectum 22, but air bubbles 60 may nevertheless exist between the balloon's outer surface 621 and the rectal inner surface 24.

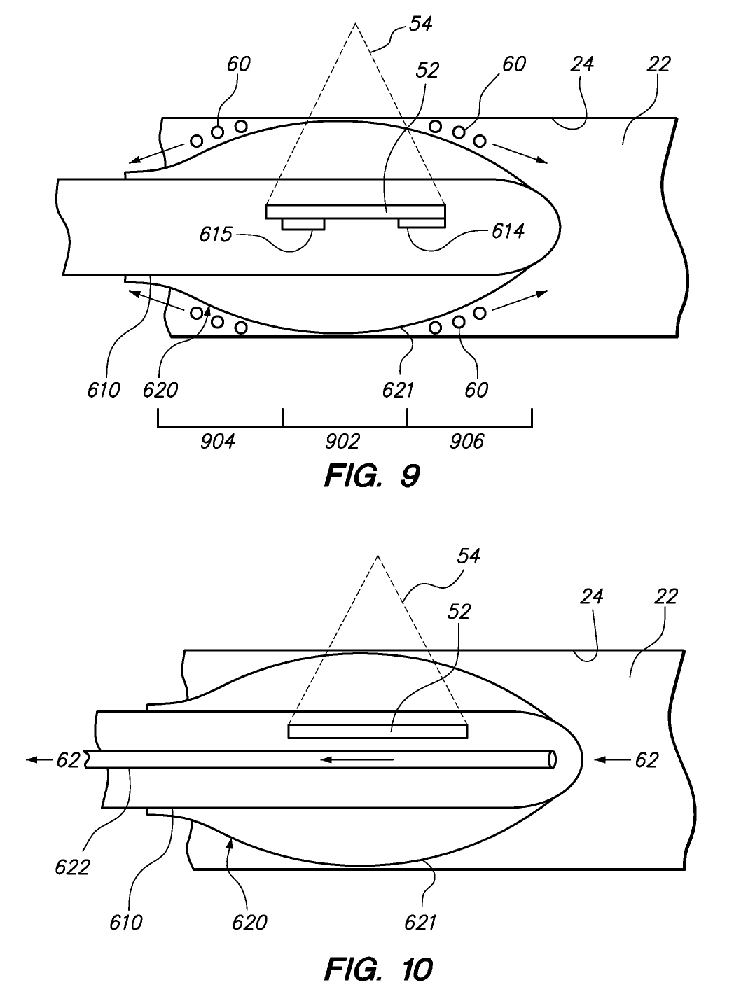

Referring to FIG. 9, after the anisotropic balloon 620 is properly positioned inside the rectum 22, the balloon 620 is inflated using, e.g., degassed water 652, or another suitable fluid or material, to a fully expanded or inflated state. In the illustrated embodiment, a middle portion 902 of the anisotropic balloon 620 initially expands, followed by expansion of proximal and distal portions 904 and 906 on each side of the middle portion 902. In an alternative embodiment, the middle portion 902 expands to a greater degree or at a faster rate relative to proximal and distal portions 904 and 906. In certain embodiments, the middle portion 902 may expand, and the proximal and distal portions 904 and 906 may be fixed and not expand at all.

Thus, during inflation, the anisotropic design and elasticity of the balloon 620 advantageously allows the balloon 620 to expand from the center and outwardly or sideways from the center towards the other portions or ends 904 and 906 of the balloon 620. As a result, the outer surface 621 of the balloon 620 pushes air bubbles 60 sideways and away from the center of the balloon 620 along the inner surface 24 of the rectum 22. This allows bubbles 60 to escape or be pushed away from the outer surface 621 that contacts the inner rectal wall 24, thereby forming a bubble-free or substantially bubble-free interface between the outer surface 621 of the balloon 620 and the inner surface 24 of the rectum 22.

It should be understood that the length of the middle portion 902 can vary, and that during inflation, different portions of the outer surface 621 of the balloon 620 may contact the inner surface 24 of the rectum 22 while pushing the air bubbles 60 away. The balloon 620 may expand sideways from the center of the balloon and outwardly in proximal and distal directions so that only the middle portion 902 engages the inner surface 24, or two or more or all of the portions 902, 904 and 906 engage the inner surface 24 and form a bubble-free or substantially bubble-free interface.

The resulting bubble-free or substantially bubble-free interface improves acoustic coupling and reduces or eliminates interference and reflections of acoustic energy 54 reflections that may otherwise result from trapped air bubbles 60 resulting from use of a known isotropic balloon (as illustrated in FIG. 5). Thus, acoustic energy 54 from the transducer 52 can be properly focused and applied to target prostate 16 tissue with greater accuracy compared to known ultrasound probes that use an isotropic balloon.

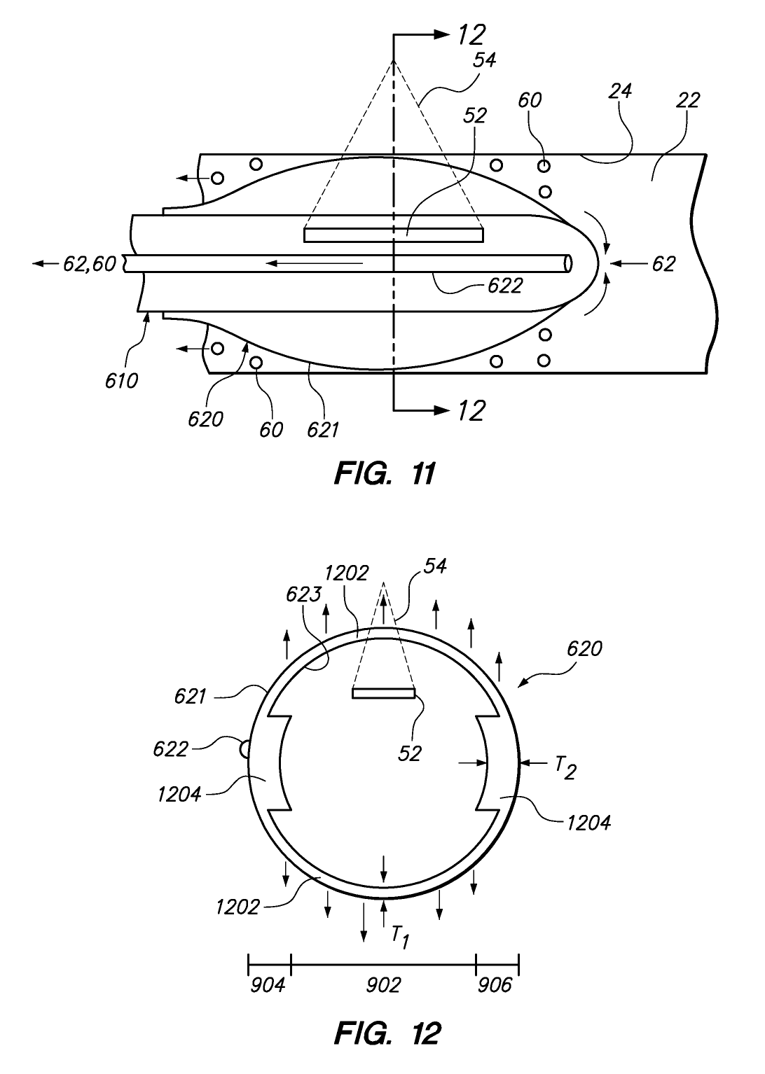

Referring to FIG. 10, a semi-rigid duct or tube 622 associated with, e.g., attached or integrated within, the anisotropic balloon 620 vents gas 62 inside the rectum 22, through the tube 622, and outside of the body. In the illustrated embodiment, the distal end of the vent tube 622 is adjacent the distal tip of the anisotropic balloon 620, however, the vent tube 622 may extend to different lengths while being capable of venting gas 62. Referring to FIG. 11, in addition to venting gas 62, air from displaced air bubbles 60 may also be vented through the tube 622. Further, a portion of the tube 622 located outside of the body can include a standard syringe interface to allow pumping of air into the rectum 22 or withdrawing of fluid from the tube 622 in the event of an obstruction within the tube 622. Additionally, although FIGS. 10 and 11 illustrate a single vent tube 622, alternative embodiments may include multiple vent tubes 622 associated with an outer surface 612 of the anisotropic balloon 620.

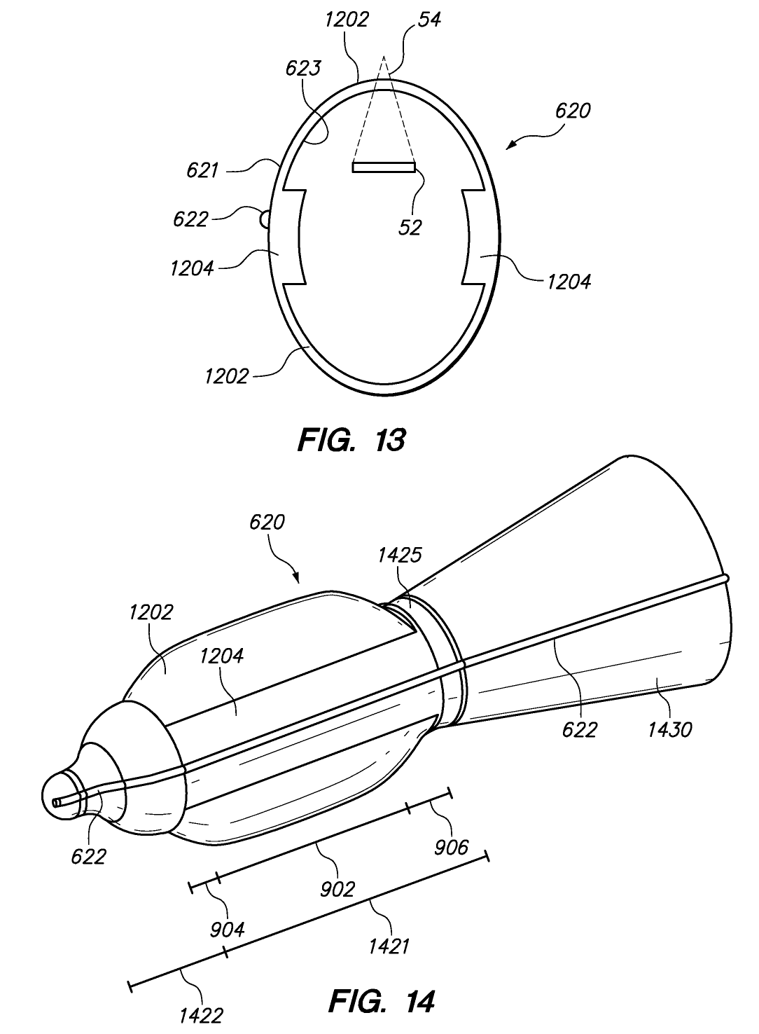

FIG. 12 is a cross-sectional view of an anisotropic balloon 620 according to one embodiment that is a multi-element or multi-section balloon having portions of different thicknesses in order to control or restrict the extent of and/or direction of expansion (illustrated by arrows) and the resulting shape of the inflated balloon 620. In one embodiment, portions 1202 of the balloon 620 are thinner than portions 1204 and expand when inflated (as shown in FIG. 13), whereas portions 1204 are rigid or semi-rigid and do not expand, or expand to a lesser degree. The ends of the portions 1202 are coupled to probe components such that air bubbles 60 are displaced from the center of the balloon 620 by the portions 1202 when the balloon is inflated (as shown in FIGS. 13 and 14). Further, since the vent tube 622 is associated with the rigid or semi-rigid portion 1204 of the balloon 620, gas venting can be achieved through the external vent tube 622 without obstructing the path of an acoustic beam 54 emitted by an transducer 52 through an expandable or flexible portion 1202 of the anisotropic balloon 610. FIGS. 12 and 13 illustrate an embodiment in which different portions 1202 and 1204 of the balloon 620 have different discrete thicknesses. In alternative embodiments, the thickness of different portions 1202 and 1204 can vary gradually. Accordingly, FIGS. 12 and 13 are provided to illustrate one manner in which embodiments can be implemented.

FIG. 14 illustrates one embodiment of an anisotropic balloon member 1420 that includes an inflatable portion 1421 and a non-inflatable rounded distal portion 1422. The inflatable portion 1421 includes portions 1202 and 1204, e.g., an inflatable portion 1202 and a non-inflatable, rigid or semi-rigid portion 1204 to provide anisotropic expansion. A coupling or sealing ring 1425 is provided at a proximal end of the balloon 620 for sealing the interior volume of the balloon 610 to prevent leakage of water from inside the balloon 620 to the environment. Further, the balloon 620 is rigidly attached to a portion of the probe body 610 in order to prevent the balloon 610 from inflating into the rectal cavity. The rigid or semi-rigid portions 1204 also restrict the direction of expansion and shape of the inflated balloon 620.

A hygienic annular member 1430 may be attached to a base or proximal end of the anisotropic balloon 620, e.g., adjacent to the coupling ring 1425, to provide a protective barrier between a physician and the patient. In the illustrated embodiment that includes the optional annular member 1430, the vent tube 622 extends along the annular member 1430, across the coupling ring 1425, and along the portions 1421 and 1422. In the illustrated embodiment, the vent tube 622 is associated with the rigid or semi-rigid portion 1204 of the balloon 620. In the illustrated embodiment, the portion 1204 is a portion of the anisotropic balloon 620 that is not expandable, whereas portion 1202 (e.g., which can be thinner than portion 1204) is expanded when the balloon 620 is inflated. This configuration allows for gas venting without obstructing the path of the acoustic beam 54 that radiates through an expandable portion 1202.

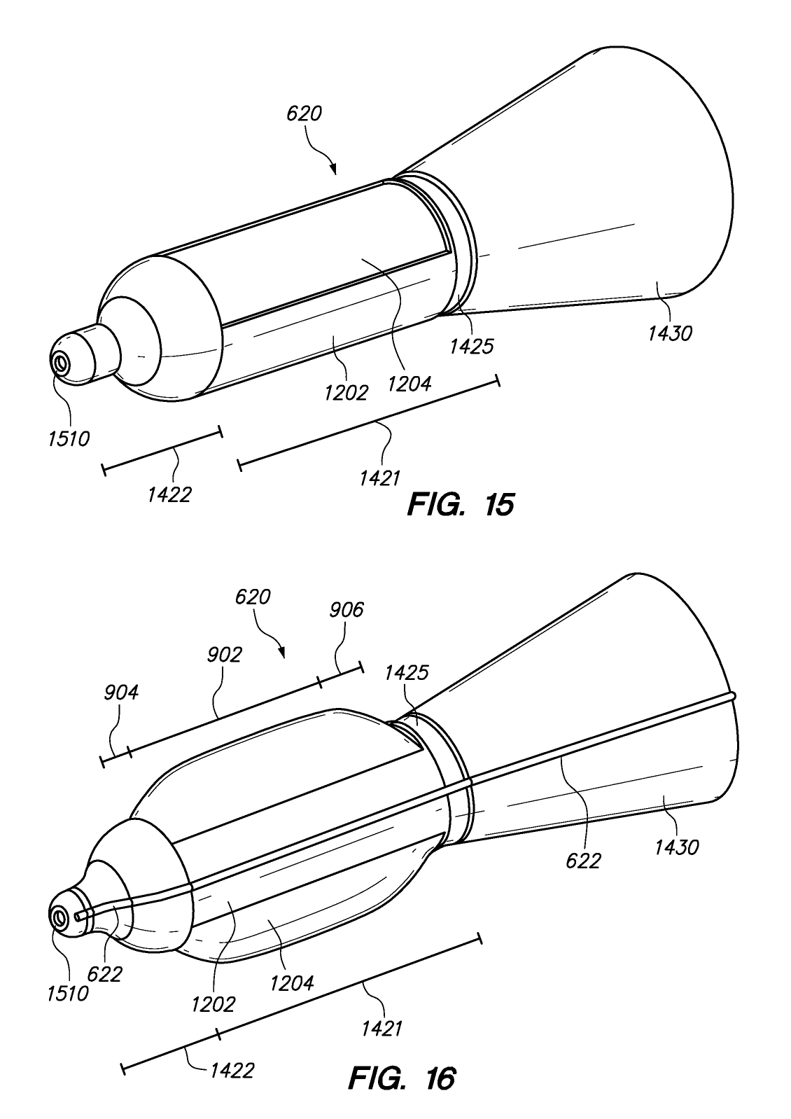

FIG. 15 illustrates an anisotropic balloon 620 according to another embodiment that includes a distal tip having valve 1510 for venting or bleeding air from within the balloon. For example, the valve 1510 can be used to vent air from within the balloon 620 during initial filling of the balloon 620 with coolant fluid. FIG. 16 illustrates another embodiment of an anisotropic balloon 620 that includes both a vent tube 622 associated with an outer surface 621 of the balloon 620 and a vent valve 1510 at distal tip of the balloon 620 for releasing air from within the balloon 620.

Referring to FIG. 17, a probe body 610 according to one embodiment that may be used with an anisotropic balloon as shown in various figures includes a substantially rigid housing or shell 1702 that defines an open space or window 1704 for the transducer 622. The distal end of the shell 1702 is configured for insertion into the balloon 610. In the illustrated embodiment, the distal tip of the shell 1702 has a shape that generally corresponds to the distal tip of the balloon 620 so that the balloon 620 may be secured over or attached to the distal tip of the probe body 610. Further, the balloon 620 can be attached to a proximal portion of the probe body 610. For example, an inner surface of the balloon 610 can mate or interface with an outer surface of the probe body 610. In this manner, the balloon 610, while being expandable in at least one direction by inflation of portions 1204, may be rigidly attached to the probe shell 1702 to prevent the balloon 610 from inflating into the rectal cavity.

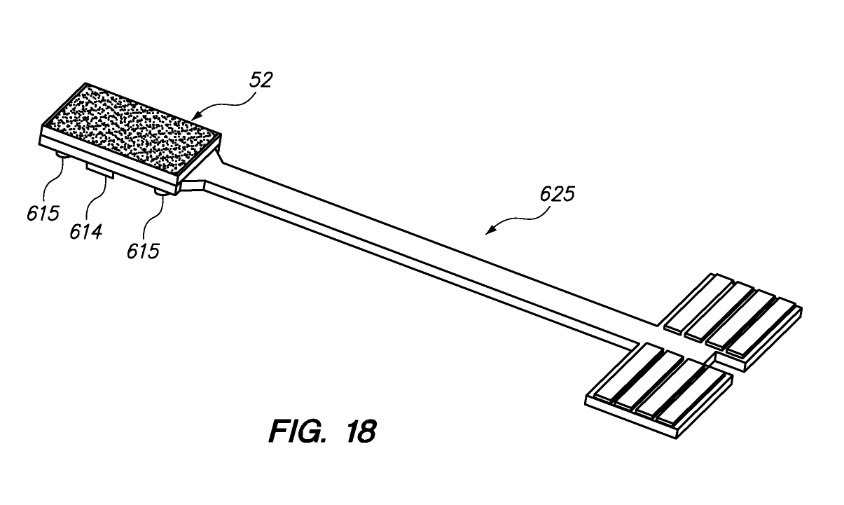

The transducer 52 is housed within the shell 1702 and may be carried by the circuit board 625 or other suitable substrate. An example of a suitable circuit board 625 is illustrated in FIG. 18. The circuit board 625 or other substrates or components carry deliver excitation or drive signals via wires or other connectors to individual elements 52a, 52b, . . . 52n of the transducer 52.

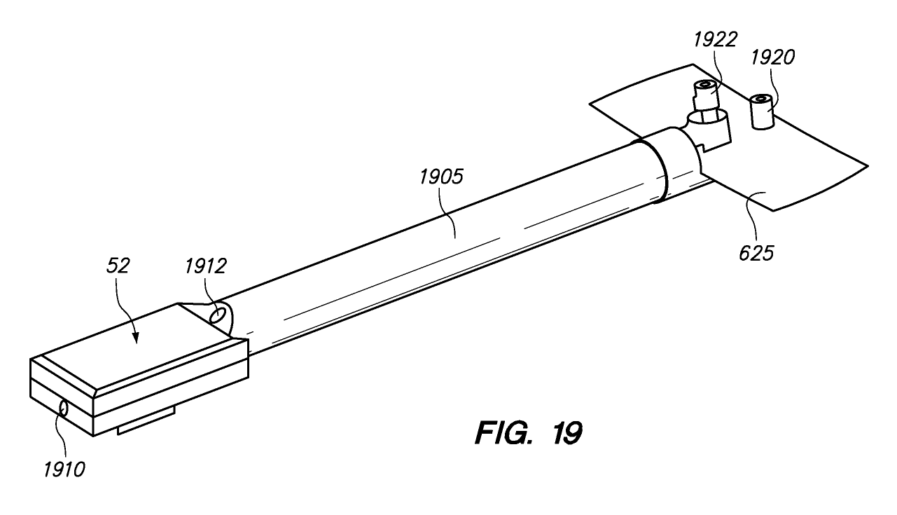

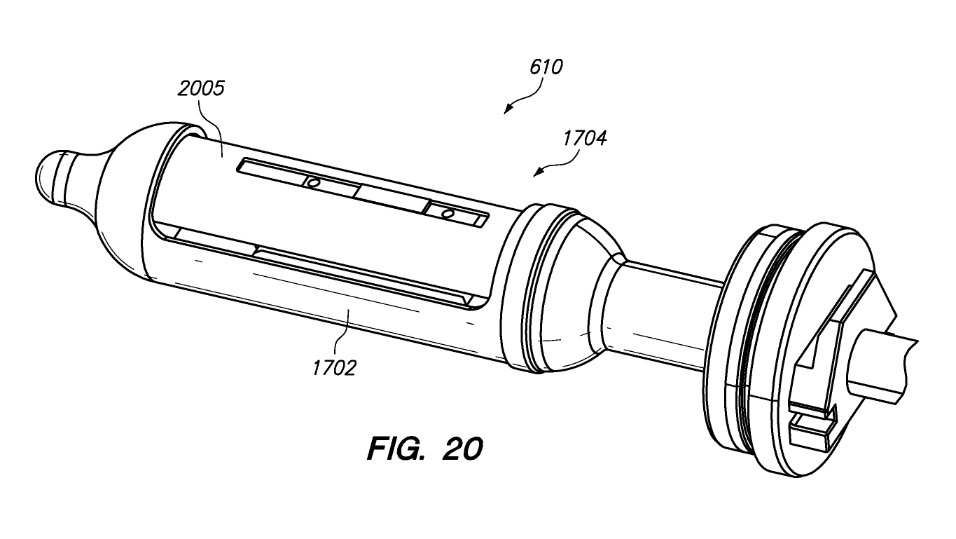

The circuit board 625 or substrate is carried by a shaft 1905 (as further illustrated in FIG. 19). The shaft 1905 and the transducer 52 carried thereby are rotatable within the shell 1702 in order to rotatably position the transducer 52 within the window 1704 when a procedure is to be performed, or away from the window 1704 to protect the transducer 52 or to prevent exposure to the transducer 52 during positioning of the probe 600 when the transducer 52 is not active. The assembly of the shaft 1905 carrying the circuit board 625, which carriers the transducer 52, is inserted into the shell 1702 of the probe body 610 as shown in FIG. 17. The probe body shell 1702 and its components can then be inserted into the anisotropic balloon 610, and the assembly of the balloon 610 and probe body 620 can then be inserted into the rectum 22.

FIG. 19 also illustrates fluid or coolant ports 1910 and 1912. A fitting 1920 provides a connection for a hose or other conduit for providing a cooling fluid through port 1910 formed in the shaft 1905 under the transducer 52 for cooling the transducer 52 and providing acoustic coupling. A fitting 1922 provides a connection for a hose or other conduit for withdrawing fluid through port 1912 formed at a distal end of the shaft 1905. This arrangement provides sufficient fluid circulation and facilitates draining of air from within the balloon 620 during filling or inflation of the balloon 620 (e.g., using the balloon 620 having an air valve 1510 as shown in FIG. 15).

Referring to FIG. 20, a probe shell 1702 according to one embodiment includes a rotatable cover 2005 that can be positioned to encapsulate and protect the transducer 52. According to one embodiment, the cover 2005 can be rotated by about 180 degrees to open and close the window 1704. During use, the assembly of the anisotropic balloon 620 and the probe body 610 can be inserted into the rectum 22, and then the shaft 1905 can be rotated (e.g., 180 degrees) which, in turn, rotates the cover 2005 and transducer 52, thereby opening the window 1704 and positioning and exposing the transducer 52 within the open window 1704 so that acoustic energy 54 emitted by the transducer 52 can be directed towards the prostate 16.

According to one embodiment, the shell 1702 can be detached from the probe body 610 and discarded after each use, and a new shell 1702 can be applied to the probe body 610 for a new patient or other procedure. The shell 1702 can also have different dimensions depending on the anatomy of the particular patient to be treated, e.g., depending on the size of the rectum 22 and/or prostate 16 of the patient.

Although particular embodiments have been shown and described, it should be understood that the above description is not intended to limit the scope of embodiments since various changes and modifications may be made without departing from the scope of the claims. For example, embodiments can be implemented using various ultrasound transducers and other probe bodies. Further, embodiments can be implemented with one of multiple vent tubes, which can be various lengths while still being capable of venting gas. Additionally, vent tubes may be a separate component attached to a balloon or formed or made integral with the balloon. Further, although embodiments are described with reference to portions of an anisotropic balloon having different thicknesses, embodiments may also be implemented using portions composed of different materials that may also have different thicknesses so that portions have different elasticity and different degrees or rates of anisotropic expansion. Thus, embodiments are intended to cover alternatives, modifications, and equivalents that fall within the scope of the claims.

What is claimed is:

1. A transrectal ultrasound probe, comprising:
a probe body carrying an ultrasound transducer;
an anisotropic balloon member defining a cavity configured for receiving the probe body; and
a tube associated with an outer surface of the anisotropic balloon member, the tube being configured for venting of rectal gas.

2. The probe of claim 1, the ultrasound transducer being a focused ultrasound transducer for treating or ablating tissue.

3. The probe of claim 1, the ultrasound transducer comprising an array of ultrasound transducer elements.

4. The probe of claim 1, the probe body defining a window for the ultrasound transducer.

5. The probe of claim 4, the probe body including a cover element that is rotatably positionable within the probe body to expose and cover the ultrasound transducer.

6. The probe of claim 1, different portions of the anisotropic balloon member having different thicknesses.

7. The probe of claim 1, wherein a thickness of middle portion of the anisotropic balloon member is less than a thickness of portions of the anisotropic balloon member adjacent the middle portion.

8. The probe of claim 7, wherein the middle portion can expand more than the adjacent portions when the anisotropic balloon member is inflated.

9. The probe of claim 1, further comprising a temperature sensor associated with the ultrasound transducer.

10. The probe of claim 9, wherein the temperature sensor is not attached to the anisotropic balloon member.

11. The probe of claim 1, the tube being attached to the outer surface of the anisotropic balloon member.

12. The probe of claim 1, the tube being conformable to a shape of the anisotropic balloon member.

13. The probe of claim 12, the tube being conformable to a non-linear outer surface of the anisotropic balloon member.

14. The probe of claim 1, wherein no portion of the tube is positioned inside the anisotropic balloon member.

15. The probe of claim 1, a distal end of the anisotropic balloon member defining a duct for venting air from within the anisotropic balloon member.

16. The probe of claim 1, further comprising a location sensor associated with the transducer.

17. The probe of claim 16, the location sensor including a plurality of magnetic resonance micro-coils.

18. A transrectal ultrasound probe, comprising:
a probe body carrying an ultrasound transducer for treating or ablating tissue;
a balloon member defining a cavity configured for receiving the probe body, wherein a thickness of a middle portion of the balloon member is less than a thickness of portions of the balloon member adjacent the middle portion;
a tube conformable with an outer surface of the balloon member and being configured for venting of rectal gas; and
a temperature sensor associated with the ultrasound transducer.

19. The probe of claim 18, wherein the middle portion can expand more than the adjacent portions when the balloon member is inflated.

20. The probe of claim 18, wherein the temperature sensor is not attached to the balloon member.

21. The probe of claim 18, the tube being attached to the outer surface of the balloon member.

22. The probe of claim 18, the tube being conformable to a non-linear outer surface of the balloon member.

23. The probe of claim 18, wherein no portion of the tube is positioned inside the balloon member.

24. The probe of claim 18, a distal end of the balloon member defining a duct for releasing air from within the balloon member.

25. A method of ablating tissue of a patient, comprising:
inserting a probe into a rectum of the patient, the probe including a probe body carrying an ultrasound transducer, a balloon member defining a cavity configured for receiving the probe body, and a tube conformable with an outer surface of the balloon member;
activating the ultrasound transducer; and
ablating tissue using ultrasound energy emitted by the ultrasound transducer.

26. The method of claim 25, further comprising venting gas in the rectum through the tube and outside of the patient.

27. The method of claim 26, further comprising venting air from within the balloon member through a duct defined by a distal end of the balloon member.
